(19) United States
(12) Patent Application Publication
(10) Pub. No.: US 2012/0251524 A1
(43) Pub. Date: Oct. 4, 2012

(54) USE OF CYTOKINE LEVELS IN INTRAVENOUS IMMUNOGLOBULIN TREATMENT OF ALZHEIMER'S DISEASE

(75) Inventors: Norman R. Relkin, Harrington Park, NJ (US); Larry Backes, Guilford, CT (US); Richard Schiff, Eastsound, WA (US)

(73) Assignees: Baxter Healthcare S.A., Glattpark (Opfikon) (CH); Baxter International Inc., Deerfield, IL (US)

(21) Appl. No.: 13/432,996

(22) Filed: Mar. 28, 2012

Related U.S. Application Data
(60) Provisional application No. 61/470,819, filed on Apr. 1, 2011.

(51) Int. Cl. A61K 39/395; C40B 30/00; G01N 27/447; H01J 49/26; G01N 33/559; G01N 33/53; A61P 25/28

(52) U.S. Cl. 424/130.1; 436/501; 506/7; 250/282; 204/456

(57) ABSTRACT

The present invention relates to the use of the level of certain cytokines in a patient's blood as an objective measure for the purpose of assessing disease progression in patients suffering from Alzheimer's disease and for the purpose of determining therapeutic effectiveness of a treatment regimen. Methods for treating Alzheimer's disease and monitoring therapeutic effectiveness are provided.

### RELATED APPLICATIONS

This application claims priority to U.S. Provisional Patent Application No. 61/470,819, filed Apr. 1, 2011, the contents of which are incorporated herein in the entirety for all purposes.

### BACKGROUND OF THE INVENTION

Alzheimer's disease is the most common form of dementia afflicting as many as 5.3 million Americans. The disease is generally believed to be caused by the accumulation of β-amyloid plaques in the brain, resulting in nerve cell death and concomitant reduction in neurotransmitters levels. Impairment in memory, cognition, reasoning, and judgment results along with the decrease in emotional stability and development of behavioral problems. The disease is progressive leading to profound mental deterioration and ultimately death.

There is no known cure for the Alzheimer's disease. Patient care primarily focuses on the management of symptoms of this disease. Disease progression in Alzheimer's patients can be monitored in terms of reduction in brain tissue volume (enlargement of ventricular volume) or continued deterioration of cognitive ability over time. Afforded by technologies such as magnetic resonance imaging (MRI), these image-based monitoring techniques are advantageous in their ease to administer and to quantify any changes in the brain condition. The recent discovery that antibodies against β-amyloid are present in human immunoglobulin preparations (e.g., intravenous immunoglobulin or IVIG) and can inhibit the neurotoxic effects of β-amyloid lead to clinical trials in Alzheimer's patients. Disease stabilization and modest improvement in cognitive ability were noted.

In 2006, there were 26.6 million Alzheimer's disease sufferers worldwide. By 2050, a predicted 1 in every 85 people will be diagnosed globally. Given the dire nature of this disease, the large patient population, and the tremendous burden on care givers, a pressing need exists for new and more effective therapeutic agents and methods. The present invention provides improvements to fulfill this and other related needs.

### BRIEF SUMMARY OF THE INVENTION

This inventions relates to the use of changes in certain cytokine level in a patient's blood to monitor the effect of a brain preserving treatment of Alzheimer's disease and to guide formulating further treatment plans.

In one aspect, the present invention provides a method for treating Alzheimer's disease in a subject in need thereof. The method comprises these sequential steps: (a) determining the amount of a cytokine in the subject's blood, thereby obtaining a baseline value of the cytokine level; (b) administering a brain preserving therapeutic agent to the subject for the purpose of treating Alzheimer's disease during a first time period; (c) determining the amount of the cytokine in the subject's blood, thereby obtaining a first intermediate value of the cytokine level; (d) comparing the intermediate value from step (c) with the baseline value from step (a); and (e) increasing administration of the brain preserving therapeutic agent in dose or frequency when step (d) indicates no increase from the baseline value to the first intermediate value, or maintaining administration of the brain preserving therapeutic agent in dose or frequency when step (d) indicates an increase from the baseline value to the first intermediate value. Typically, step (a) or an equivalent step of quantifying the amount of the cytokine is performed by determining the cytokine level in a blood sample taken from the subject. Such a sample may be a whole blood, serum, or plasma sample.

In some cases, steps (b) to (d) are further repeated at least once and in each repeat the latest intermediate value is compared with the second latest intermediate value to determine future administration of the therapeutic agent in the same manner as step (e). In some cases, when step (d) during any repeat indicates no increase from one intermediate value to its subsequent intermediate value, and the administration of the brain preserving therapeutic agent is increased in dose or frequency, the method further comprises the steps of: (f) determining the cytokine level in the subject's blood after an additional time period during which the therapeutic agent is administered to the subject, thereby obtaining additional intermediate value of the cytokine level; (g) comparing the additional intermediate value with its previous intermediate value; and (h) discontinuing further administration of the therapeutic agent when step (g) indicates no increase from the previous intermediate value to the additional intermediate value, or maintaining administration of the brain preserving therapeutic agent in dose or frequency when step (g) indicates an increase from the previous intermediate value to the additional intermediate value.

In some cases, the first time period is 3 months, 6 months, 9 months, 12 months, or 18 months. In other cases, the second or subsequent time period is 3 months, 6 months, 9 months, 12 months, or 18 months. In some cases, the cytokine monitored in the claimed method is IL-1A, IL-4, IL-5, IL-6, IL-8, IL-13, VEGF, G-CSF, EGF, IL-12p70, IL-17, MIP-1A, MIP-1B, or IP-10, although more than one may be monitored for the same time period.

In some cases, the therapeutic agent is an intravenous immunoglobulin (IVIG) composition, which may be administered according to different schedule, such as at about 0.2 to 2 grams per kg body weight of the subject per month, at a frequency of once a week, twice a week, once a month, or twice a month. In one particular example, the IVIG composition is administered at about 0.4 gram per kg body weight of the subject twice a month. Further, the IVIG composition may be administered by different routes, such as subcutaneously, intravenously, and intranasally.

In some embodiments of the method described above, any of the steps where the level of a cytokine is determined, such as step (a), (c), or (f), may be performed by way of an immunological assay, which may include the use of microfluidic devices such as microarray protein chips, detection by gel electrophoresis and western blot analysis using specific antibodies, and other antibody-based assays such as ELISA. In addition, the step of determining the cytokine level may be performed by any one of mass spectrometry-based methods.

In another aspect, the present invention provides a method for assessing efficacy of a therapy intended for treating Alzheimer's disease. The method comprises these steps: (a) determining the average level of a cytokine in the blood of subjects suffering from Alzheimer's disease but not receiving the therapy, thereby obtaining a non-therapeutic level of the cytokine; (b) determining the average level of the cytokine in the blood of subjects suffering from Alzheimer's disease and receiving the therapy, thereby obtaining a therapeutic level of the cytokine; and (c) comparing the therapeutic level with the non-therapeutic level, thereby determining the efficacy of the therapy, wherein the therapy is deemed effective when the therapeutic level is higher than the non-therapeutic level, and the therapy is deemed ineffective when the therapeutic level is equal to or lower than the non-therapeutic level. Typically, steps (a) and (b) or any equivalent steps of quantifying the amount of the cytokine are performed by determining the average cytokine level in blood samples taken from Alzheimer's patients. Such samples may be whole blood, serum, or plasma samples.

In some cases, the cytokine is IL-1A, IL-4, IL-5, IL-6, IL-8, IL-13, VEGF, G-CSF, EGF, IL-12p70, IL-17, MIP-1A, MIP-1B, or IP-10, although more than one may be monitored at the same time. In some cases, the therapy is administration of an intravenous immunoglobulin (IVIG) composition, which may be administered according to different schedule, such as at about 0.2 to 2 grams per kg body weight of the subject per month. In some cases, the administration frequency may be once a week, twice a week, once a month, or twice a month. In one particular example, the IVIG composition is administered at about 0.4 gram per kg body weight of the subject twice a month. In some cases, the cytokine level in step (a) or (b) is determined over a time period of about 3 months, 6 months, 9 months, 12 months, or 18 months. Further, the IVIG composition may be administered by different routes, such as subcutaneously, intravenously, and intranasally.

In some cases, any of the steps where the level of a cytokine is determined, such as step (a) or (b), may be performed by way of an immunological assay, which may include the use of microfluidic devices such as microarray protein chips, detection by gel electrophoresis and western blot analysis using specific antibodies, and other antibody-based assays such as ELISA. In addition, the step of determining the cytokine level may be performed by any one of mass spectrometry-based methods.

Although multiple subjects (e.g., subjects including at least 5 individuals) are often used in the above-described methods for assessing therapeutic efficacy of an anti-Alzheimer's therapy, such methods may also be practiced on a single individual to determine whether any particular therapeutic modality or treatment schedule is effective for that individual. More specifically, the method for determining the efficacy of a therapy intended for treating Alzheimer's disease in a subject includes these steps: (a) determining the level of a cytokine in a blood sample taken from a subject who is suffering from Alzheimer's disease but has not received the therapy, thereby obtaining a baseline level of the cytokine; (b) determining the level of the cytokine in a blood sample taken from the subject after having received the therapy for a time period, thereby obtaining a therapeutic level of the cytokine; and (c) comparing the therapeutic level with the baseline level, thereby determining the efficacy of the therapy in the subject. The therapy is deemed effective for the subject during the time period when the therapeutic level is higher than the baseline level, and the therapy is deemed ineffective for the subject during the time period when the therapeutic level is equal to or lower than the baseline level. In some embodiments, the cytokine is IL-1A, IL-4, IL-5, IL-6, IL-8, IL-13, VEGF, G-CSF, EGF, IL-12p70, IL-17, MIP-1A, MIP-1B, or IP-10. In some embodiments, the therapy is administration of an intravenous immunoglobulin (IVIG) composition, which may be administered by various means, including subcutaneously and intravenously. In some embodiments, the IVIG composition is administered at about 0.2 to 2 grams per kg body weight of the subject per month. For example, the IVIG composition is administered once a week, twice a week, once a month, or twice a month. In one particular example, the IVIG composition is administered at about 0.4 gram per kg body weight of the subject twice a month. In some embodiments, the time period in step (b) is about 3 months, 6 months, 9 months, 12 months, or 18 months. Once a determination of therapeutic efficacy is made, the physician treating the patient(s) should either maintain administration of the therapeutic agent in dose or frequency when the therapy is found effective; or increase administration of the therapeutic agent in dose or frequency when the therapy is found ineffective. After at least one additional round, optionally two or more rounds, of increasing administration/assessing efficacy, the physician should discontinue treatment in the patient(s) if the therapy remains ineffective as determined by any one of the efficacy-assessing methods described above.

### BRIEF DESCRIPTION OF THE DRAWINGS

FIG. 1: correlation among changes in plasma cytokines in AD patients after receiving IVIG treatment for 6 months.

FIG. 2: no change in plasma level of most cytokines in AD patients after receiving IVIG treatment for 6 months.

FIG. 3: three cytokines, IL-17, MIP-1A, and IL-12p70, showed a trend of significant increase in their plasma level in AD patients after receiving IVIG treatment for 6 months.

FIG. 4: nine cytokines, IL-1A, IL-4, IL-5, IL-6, IL-8, IL-13, VEGF, G-CSF, and EGF, showed highly significant changes in their plasma level in AD patients after receiving IVIG treatment for 6 months.

FIG. 5: changes in cytokine plasma level were IVIG dose dependent in AD patients after receiving IVIG treatment for 6 months.

FIG. 6: correlation between clinical outcomes and plasma cytokine levels in AD patients after receiving IVIG treatment for 6 months.

### DEFINITIONS

“Alzheimer's disease (AD)” is a common form of dementia typically observed among people over 65 years of age, although the early-onset type may occur much earlier. An incurable, irreversible, progressive brain disease, Alzheimer's disease is diagnosed based on certain common symptoms. In the early stages, the most commonly recognized symptom of AD is memory loss, such as difficulty in remembering recently learned facts. A physician will typically confirm the diagnosis of AD with behavioral assessments and cognitive tests, often followed by a brain scan. As the disease advances, further symptoms will become evident, including confusion, irritability and aggression, mood swings, language breakdown, long-term memory loss, and the general withdrawal of the patients as their senses decline. As used herein, a patient suffering from Alzheimer's disease or AD may be afflicted with any variation of the brain disorder and at any stage of the condition as diagnosed according to the currently used diagnostic criteria.

As used herein, “cytokines” encompass low molecular weight proteins secreted by various cells in the immune system that act as signaling molecules for regulating a board range of biological processes within the body at the molecular and cellular levels. “Cytokines” include individual immunomodulating proteins that fall within the class of lymphokines, interleukins, or chemokines. For instance, IL-1A and IL-1B are two distinct members of the human interleukin-1 (IL-1) family. Mature IL-1A is a 18 kDa protein, also known as fibroblast-activating factor (FAF), lymphocyte-activating factor (LAF), B-cell-activating factor (BAF), leukocyte endogenous mediator (LEM), etc. IL-4 is a cytokine that induces T helper-2 (Th2) cell differentiation, and is closely related to and has similar functions to IL-13. IL-5 is produced by Th2 cells and mast cells. It acts to stimulate B cell growth and increase immunoglobulin secretion. It is also involved in eosinophil activation. IL-6 is an interleukin that can act as either a pro-inflammatory or anti-inflammatory cytokine. It is secreted by T cells and macrophages to stimulate immune response to trauma or other tissue damage leading to inflammation. IL-6 is also produced from muscle in response to muscle contraction. IL-8 is a chemokine produced by macrophages and other cell types such as epithelial cells and endothelial cells, and acts as an important mediator of the immune reaction in the innate immune system response. IL-12 is involved in the differentiation of naïve T cells to T helper (Th1 or Th2) cells. A heterodimeric cytokine, IL-12 is formed after two subunits encoded by two separate genes, IL-12A (p35) and IL-12B (p40), dimerize following protein synthesized. IL-12p70 indicates this heterodimeric composition. IL-13, a cytokine secreted by many cell types especially Th2 cells, is an important mediator of allergic inflammation and disease. IL-17 is a cytokine produced by T helper cells and is induced by IL-23, resulting in destructive tissue damage in delayed-type reactions. IL-17 functions as a pro-inflammatory cytokine that responds to the invasion of the immune system by extracellular pathogens and induces destruction of the pathogen's cellular matrix. IP-10, or Interferon gamma-induced protein 10 kDa, is also known as C-X-C motif chemokine 10 (CXCL10) or small-inducible cytokine B10. A small cytokine belonging to the CXC chemokine family, IP-10 is secreted by several cell types (including monocytes, endothelial cells and fibroblasts) in response to IFN-γ. Macrophage Inflammatory Proteins (MIP) belong to the family of chemokines. There are two major forms of human MIP, MIP-1α and MIP-1β, which are also known as chemokine (C-C motif) ligand 3 (CCL3) and CCL4, respectively. Both are produced by macrophages following stimulation with bacterial endotoxins. Granulocyte colony-stimulating factor (G-CSF or GCSF), also known as colony-stimulating factor 3 (CSF 3), is a colony-stimulating factor hormone. G-CSF is a glycoprotein, growth factor, and cytokine produced by a number of different tissues to stimulate the bone marrow to produce granulocytes and stem cells. G-CSF also stimulates the survival, proliferation, differentiation, and function of neutrophil precursors and mature neutrophils. Epidermal growth factor or EGF is a growth factor that plays an important role in the regulation of cell growth, proliferation, and differentiation by binding with high affinity to its receptor EGFR. Vascular endothelial growth factor (VEGF) is a -family of growth factors that are important signaling proteins involved in both vasculogenesis (the de novo formation of the embryonic circulatory system) and angiogenesis (the growth of blood vessels from pre-existing vasculature).

“Intravenous immunoglobulin” or “IVIG” refers to a blood product that contains the pooled immunoglobulin G (IgG) immunoglobulins from the plasma of a large number (often more than a thousand) of blood donors. Typically containing more than 95% unmodified IgG, which has intact Fc-dependent effector functions, and only trace amounts of immunoglobulin A (IgA) or immunoglobulin M (IgM), IVIGs are sterile, purified IgG products used in treating certain medical conditions. Although the word “intravenous” typically indicates administration by intravenous injection, the term “IVIG” or “IVIG composition” as used in this patent application also encompasses an IgG composition that is formulated for administration by additional routes, including subcutaneous or intranasal administration.

The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains.

The term “antibody” also refers to antigen- and epitope-binding fragments of antibodies, e.g., Fab fragments, that can be used in immunological affinity assays. There are a number of well characterized antibody fragments. Thus, for example, pepsin digests an antibody C-terminal to the disulfide linkages in the hinge region to produce F(ab)′₂, a dimer of Fab which itself is a light chain joined to VH-CH₁ by a disulfide bond. The F(ab)′₂ can be reduced under mild conditions to break the disulfide linkage in the hinge region thereby converting the (Fab′)₂ dimer into an Fab′ monomer. The Fab′ monomer is essentially a Fab with part of the hinge region (see, e.g., Fundamental Immunology, Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody also includes antibody fragments either produced by the modification of whole antibodies or synthesized using recombinant DNA methodologies.

“An increase” or “a decrease” as used herein refers to a positive or negative change in quantity from a comparison control (such as the baseline value of a cytokine level), respectively. An increase is typically at least 10%, or at least 20%, or 50%, or 100%, and can be as high as at least 2-fold or at least 5-fold or even 10-fold. Similarly, a decrease is typically at least 10%, or at least 20%, 30%, or even as high as 50% or more in reduction from the level of the comparison control.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the peptide or used to detect antibodies specifically reactive with the peptide.

The term “blood” as used herein refers to a blood sample or preparation from a subject being tested for cytokine level and for assessing the progression of the subject's Alzheimer's Disease. A “blood sample” in this application may refers to any fraction of blood from which at least 95% of all cells present in whole blood have been removed, and encompasses fractions such as serum and plasma as conventionally defined. Blood samples obtained from different individuals or from the same individual but at different time points following the same processing steps are referred to as “the same type of blood samples.”

The phrase “specifically binds,” when referring to a protein or peptide, refers to a binding reaction that is determinative of the presence of the protein in a heterogeneous population of proteins and other biologics. Thus, under designated immunoassay conditions, the specified binding agent (e.g., an antibody) binds to a particular protein at least two times the background and does not substantially bind in a significant amount to other proteins present in the sample. Specific binding to an antibody under such conditions may require an antibody that is selected for its specificity for a particular protein or a protein but not its similar “sister” proteins. For example, antibodies may be raised to specifically bind interferon-α (IFN-α) but not interferon-β (IFN-β) protein. In the alternative, antibodies can be raised and selected to specifically bind IFN-β protein but not IFN-α protein. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background.

### DETAILED DESCRIPTION OF THE INVENTION

### I. Introduction

Although there is no cure in Alzheimer's Disease (AD), several brain preserving therapeutic methods, which can slow or even halt mental deterioration associated with AD, are currently being studied for use in the treatment and alleviation of AD symptoms. Intravenous immunoglobulin (IVIG) immunotherapy is one of such therapies. IVIG treatment has been shown to reduce the rated of cognitive deterioration among AD patients and this effect has been observed as varying with IVIG dosage. Although various cognitive tests are available for assessing a patient's brain function, therefore useful for assessing the effectiveness of a therapeutic regimen for treating AD, alternative methods, especially ones that are easy to administer, are desired for quick and objective means to monitor any changes in AD patients' cognitive ability in response to a brain preserving therapy. The present inventor has observed statistically significant changes in certain cytokine level in the blood of AD patients receiving brain preserving treatment after some time period, e.g., 3 months, 6 months, or 12 months. Because these changes correlate with AD patients' brain functions as measured by the cognitive tests and are IVIG dose dependent, the cytokine monitoring method is therefore relatively faster and more objective for determining efficacy of a brain preserving therapy in AD patients. In addition, such cytokine signals may be used to determine the efficacy of brain preserving therapy in AD patients more quickly than cognitive testing, as clinically determinative differences in cognitive testing can be difficult in monitoring decline in an individual patient over shorter time periods (e.g., 3 months, 6 months, or 12 months) due to variability and imprecision of the cognitive testing methods.

### II. IVIG Treatment of Alzheimer's Disease

### A. Patients to Receive Treatment

Patients to receive treatment by the IVIG composition (or other anti-Alzheimer brain preserving therapeutic agents) according to the present invention are diagnosed to suffer from Alzheimer's disease. The onset of Alzheimer's disease is usually gradual, and it is slowly progressive. Problems with memory, particularly short-term memory, are common early in the course of Alzheimer's disease. Mild personality changes, such as less spontaneity, apathy, and a tendency to withdraw from social interactions, may also occur early in the illness. As the disease progresses, problems in abstract thinking and in other intellectual functions develop. The patient may begin to have trouble with figures when working on bills, with understanding what is being read, or with organizing the day's work. Further disturbances in behavior and appearance may also be seen at this point, such as agitation, irritability, quarrelsomeness, and a diminishing ability to dress appropriately. Later in the course of the disorder, affected individuals may become confused or disoriented about what month or year it is, be unable to describe accurately where they live, or be unable to name a place being visited. Eventually, patients may wander, become unable to engage in conversation, erratic in mood, uncooperative, and lose bladder and bowel control. In late stages of the disease, persons may become totally incapable of caring for themselves. Death can then follow, perhaps from pneumonia or some other problem that occurs in severely deteriorated states of health. Those who develop the disorder later in life more often die from other illnesses (such as heart disease) rather than as a consequence of Alzheimer's disease.

The clinical criteria for diagnosing Alzheimer's disease are well known to a practicing physician. Alzheimer's disease is diagnosed when: (1) a person has sufficient cognitive decline to meet criteria for dementia; (2) the clinical course is consistent with that of Alzheimer's disease; and (3) no other brain diseases or other processes are better explanations for the dementia. Other causes for the cognitive problems must be ruled out before a diagnosis of Alzheimer's disease can be properly made. They include neurological disorders such as Parkinson's disease, cerebrovascular disease and strokes, brain tumors, blood clots, and multiple sclerosis, infectious diseases of the central nervous system, side effects of medications, psychiatric disorders, substance abuse, metabolic disorders, trauma, toxic factors, etc. In short, a comprehensive clinical evaluation is essential in arriving at the correct diagnosis. Such an evaluation should include at least three major components; (1) a thorough general medical workup; (2) a neurological examination including testing of memory and other functions of thinking; and (3) a psychiatric evaluation to assess mood, anxiety, and clarity of thought. In addition, imaging of the brain is sometimes used for evaluation purposes. Frequently used techniques for imaging include non-contrast CT scan and MRI. Other imaging procedures (such as SPECT, PET, and fMRI) can provide information of brain function (functional neuroimaging) but are less often used.

For the purpose of practicing the method of this invention, Alzheimer patients receiving anti-Alzheimer treatment (e.g., IVIG administration) are typically in the relatively early stages of the disease progression with mild to moderate symptoms, such that their improvement from the therapeutic agent will be easier to determine and thus their future treatment plan can be properly adjusted. In the some cases, individuals suspected of beginning to develop Alzheimer's disease or considered at risk of developing this disease may also receive such treatment, so that their progression towards onset of the disease may be halted or reversed, or their risk of developing the disease may be diminished or eliminated. In other words, the anti-Alzheimer treatment (e.g., IVIG administration) can be applied as a method of preventing Alzheimer's disease or inhibiting or delaying the onset of the disease in at-risk individuals with no or only suspected symptoms.

In some cases a therapeutic agent intended for treating Alzheimer's disease is assessed for its efficacy, in which cases Alzheimer's patients are placed in treatment and non-treatment groups for comparison purposes, for example, to demonstrate any change in the level of one or more cytokines found in patient blood attributable to the effects of the therapeutic agent. Patients assigned to the two groups would preferably have overall reasonably matched characteristics such as age, gender, medical history, ethnic background, education level, severity of their Alzheimer's disease, etc.

### B. IVIG Administration

As routinely practiced in the modern medicine, sterilized preparations of concentrated immunoglobulins (especially IgGs) are used for treating medical conditions that fall into these three main classes: immune deficiencies, inflammatory and autoimmune diseases, and acute infections. One commonly used IgG product, intravenous immunoglobulin or IVIG, is formulated for intravenous administration. Although concentrated immunoglobulins may also be formulated for administration by other routes (e.g., subcutaneous or intranasal administration), for ease of discussion, such alternatively formulated IgG compositions are also included in the term “IVIG” or “IVIG composition” in this application. IVIG products suitable for use in practicing this invention may be obtained from a number of commercial suppliers, including Baxter BioScience, Talecris Biotherapeutics, Grifols USA, Octapharma USA, and ZLB Behring.

To successfully treat a disease or condition, a therapeutic agent must be administered in an effective amount. The term “effective amount” refers to an amount of a therapeutic agent, such as an IVIG preparation, that results in a detectable improvement or remediation of a medical condition being treated in the subject (e.g., Alzheimer's disease). An effective amount to be administered to the subject can be determined by a physician with consideration of individual differences in age, weight, disease severity, dose and frequency of administration, and individual response to the therapy. In certain embodiments, an IVIG product can be administered to a subject within the range of about 0.2 g/kilogram of patient body weight to about 4 g/kilogram body weight each time, and the frequency of administration may range from twice a week, once a week, twice a month, once a month, or once every other month. One exemplar dose range of IVIG is between about 0.1 to about 1 or about 0.2 to about 0.8 g/kg patient body weight, typically administered at the frequency of twice a month or once a month. For instance, IVIG is administered to some Alzheimer's patients at the dose of 0.2, 0.4, or 0.8 g/kg body weight according to a twice-a-month schedule. In other cases, IVIG is administered at the dose of 0.2, 0.4, or 0.8 g/kg body weight according to a once-a-month schedule.

The duration of IVIG treatment for Alzheimer's disease can vary: it may be as short as 3 or 6 months, or may be as long as 18 months, 2 years, 5 years, or 10 years. In some cases, the IVIG treatment may last the remainder of a patient's natural life. Effectiveness of the IVIG treatment may be assessed during the entire course of administration after a certain time period, e.g., every 3 months or every 6 months for an 18-month treatment plan. In other cases, effectiveness may be assessed every 9 or 12 months for a longer treatment course. The administration schedule (dose and frequency) may be adjusted accordingly for any subsequent administration. This scheme of assessment and adjustment need not be limited to the IVIG treatment of Alzheimer's disease: any other therapeutic brain preserving agent used or proposed for Alzheimer's disease treatment may be analyzed and followed in the same or similar manner.

### III. Monitoring Cytokine Level and Assessing Therapeutic Efficacy

The present inventors discovered that changes in the level of certain cytokines found in the blood of AD patients receiving IVIG treatment correlates closely with their response to IVIG treatment. More specifically, therapeutic intervention IVIG administration showed a significant increase in the plasma level of several cytokines, which correlates to improvement in cognitive function as indicated by neuropsychological evaluation. Such increase in plasma cytokine levels therefore serves as a useful indicator of therapeutic efficacy. On the other hand, a lack of change or a decrease in the plasma cytokine levels following a therapeutic regimen indicates that the particular therapeutic regimen is ineffective, either due to inadequate administration dosage and/or frequency (which may suggest an increase of dosage and/or frequency in a subsequent treatment period) or due to an inherent lack of efficacy of this regiment for treating AD (which may suggest termination of the treatment). The commonly used methods for assessing a person's cognitive ability are time-consuming to administer and rely on the administrator's subjective judgment in the analysis. In comparison, changes in cytokine levels in a patient's blood can be readily detected and quantified by immunoassays or mass spectrometry-based methods. Monitoring cytokine levels therefore provides a far more objective and reliable standard for assessing an AD patient's response to IVIG treatment, and can provide an indication of response to the treatment that can be more quickly ascertained.

For example, after a period of time during which an AD patient was receiving a brain preserving therapy (such as IVIG administration), the effectiveness of the therapy is assessed by measuring the patient's plasma level of any one or more of the following cytokines: IL-1A, IL-4, IL-5, IL-6, IL-8, IL-13, VEGF, G-CSF, EGF, IL-12p70, IL-17, MIP-1A, MIP-1B, or IP-10. An increase of the blood or plasma level of the cytokine(s) indicates that the therapy has been effective, whereas a lack of change or a decrease in the blood or plasma level suggests that the therapy has been ineffective. Although each of these cytokines may individually provide a valid indication of therapeutic efficacy, typically multiple cytokine levels are monitored for a more reliable efficacy assessment. For instance, one may monitor the levels of cytokines IL-4, IL-6, and IL-13, optionally further including IL-1A, IL-5, IL-8, VEGF, GCSF, and EGF levels. Additionally, the plasma levels of IL-17, MIP-1A, and IL-12p70 can be monitored for this purpose. MIP-1B is yet another marker to be measured to provide an indication of therapeutic efficacy.

### A. Obtaining Samples

The first step of practicing the present invention is to obtain a blood sample from a subject being tested, e.g., a serum or plasma sample, from a patient suffering from Alzheimer's disease. Samples of the same type should be taken from both a control group (AD patients not receiving any type of brain preserving therapy) and a treatment group (AD patients receiving a brain preserving therapy, such as IVIG administration). Standard procedures routinely employed in hospitals or clinics are typically followed for this purpose. For example, collection of blood samples from a patient is performed on a daily basis in a medical office. An appropriate amount of sample, e.g., between 5 to 20 ml of peripheral blood, is collected and maybe stored according to standard medical laboratory testing procedure prior to further preparation.

For the purpose of monitoring disease progression and assessing therapeutic efficacy in AD patients receiving a brain preserving therapy, individual patient's blood samples may be taken at different time points before, during, and after the course of the therapy, such that the level of one or more relevant cytokines can be measured to provide information indicating the state of disease and to offer guidance for modifying future therapeutic regimen. For instance, when a patient's pertinent cytokine level is not observed to increase over a period of time upon receive the therapy, the attending physician may increase the administration dosage and/or frequency for the next treatment period, whereas when an increase is observed, the administration dosage and/or frequency may be maintained. Such monitoring and assessment may be performed repeatedly during several time periods (e.g., every 3 months, 6 months, 9 months, or every 12 months). In some cases, if continued increase of administration dosage and/or frequency over two or more treatment periods does not lead to any increase in patient's blood cytokine level, the physician may conclude that this particular type of therapy is not effective or suitable for treating the patient's AD and therefore terminate the treatment.

### B. Preparing Samples for Cytokine Detection

The serum or plasma of a blood sample from a subject is suitable for the present invention and can be obtained by well known methods. For example, a blood sample can be placed in a tube containing EDTA or a specialized commercial product such as Vacutainer SST (Becton Dickinson, Franklin Lakes, N.J.) to prevent blood clotting, and plasma can then be obtained from whole blood through centrifugation. On the other hand, serum is obtained through centrifugation following blood clotting. Centrifugation is typically conducted at an appropriate speed, e.g., 1,500-3,000×g, in a chilled environment, e.g., at a temperature of about 4-10° C. Plasma or serum may be subject to additional centrifugation steps before being transferred to a fresh tube for measuring the level of a particular cytokine in the amount of protein. In some cases, the amount of mRNA may also be used to indicate the presence and quantity of a cytokine protein in the patient's blood.

In certain applications of this invention, plasma or serum may be the preferred sample types. In other applications of the present invention, whole blood may be preferable.

### C. Determining the Protein Level of a Cytokine

A protein of any particular identity, such as a cytokine among those identified above, can be detected using a variety of immunological assays. In some embodiments, a sandwich assay can be performed by capturing the cytokine protein from a test sample with an antibody having specific binding affinity for the cytokine. The cytokine then can be detected with a labeled antibody having specific binding affinity for it. Such immunological assays can be carried out using microfluidic devices such as microarray protein chips. Cytokines can also be detected by gel electrophoresis (such as 2-dimensional gel electrophoresis) and western blot analysis using specific antibodies. Alternatively, standard immunohistochemical techniques can be used to detect a cytokine protein, using the appropriate antibodies. Both monoclonal and polyclonal antibodies (including antibody fragment with desired binding specificity) can be used for specific detection of cytokine proteins. Such antibodies and their binding fragments with specific binding affinity to a particular cytokine can be generated by known techniques.

Other methods may also be employed for measuring cytokine level in practicing the present invention. For instance, a variety of methods have been developed based on the mass spectrometry technology to rapidly and accurately quantify target proteins even in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res. 2009 February; 8(2):787-797.

### IV. Establishing a Comparison Control

In order to establish a control value of cytokine level, a group of subjects who have received a diagnosis of Alzheimer's disease is first to be selected. These individuals may optionally have the same gender, same or similar age, biological features (e.g., ethnic background), and/or medical history to be matched with the study group (AD patients to receive a brain preserving therapy). Also, the neurological and/or mental health status of the selected individuals in the control group should be examined and generally matched with the study group by well established, routinely employed methods.

Furthermore, the selected individuals in the control group should be of a reasonable size, such that the average level of a cytokine obtained from the group can be reasonably regarded as representative of the average level of this cytokine among individuals who suffer from Alzheimer's disease of a certain disease stage but have received and are receiving no anti-Alzheimer therapy. Preferably, the selected group includes at least 10 subjects. Typically, an average level of a given cytokine is established for each distinct type of sample.

Once an average control value is established for the level of a cytokine based on the individual values found in each individual of the selected group, this value is considered a standard for the cytokine level for this type of sample. For instance, a cytokine level found in a plasma sample should be used to compare with a control value of plasma cytokine level only.

### EXAMPLES

The following examples are provided by way of illustration only and not by way of limitation. Those of skill in the art will readily recognize a variety of non-critical parameters that could be changed or modified to yield essentially the same or similar results.

### Example 1

### Plasma Cytokine Changes after Intravenous Immunoglobulin (IVIG) Treatment in Patients with Alzheimer's Disease (AD)

Objectives:

(1) To explore changes in plasma cytokine levels associated with administration of IVIG to AD patients; (2) To correlate cytokine changes with clinical outcomes in a placebo-controlled, randomized Phase 2 study of Gammagard IVIG for mild to moderate AD.

Methods:

Plasma specimens were drawn from all subjects in the Phase 2 study of IVIG for mild to moderate AD. Plasma samples were drawn by venous phlebotomy prior to infusions at baseline and 6 months. The study was carried out with informed consent.

The blood draws were obtained prior to the first and last infusions to avoid the potentially confounding effects of the acute fluxes in cytokines that are reported to follow IVIG infustions.

Levels of selected cytokines and chemokines were analyzed using assays optimized for the Luminex platform. Appropriate standards and duplicate measurements were employed to promote accuracy. All reported data represent the mean of at least two measurements.

Cytokine data were rendered as a percent change from baseline to 6 months of treatment. Statistical significance of changes were established using a 2-tailed Student's T-test and correlation analyses were carried out using the Data Analysis statistics package in Excel 2007.

Results:

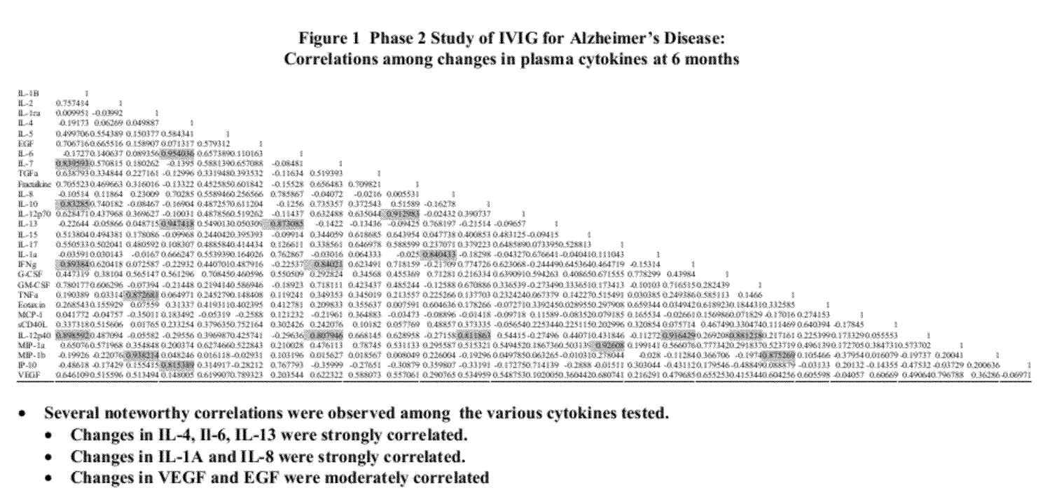

Shown in FIGS. 1-6 and described in detail below.

As indicated in FIG. 1, several noteworthy correlations were observed among the various cytokines tested: changes in IL-4, IL-6, and IL-13 were strongly correlated; changes in IL-1A and IL-8 were strongly correlated; changes in VEGF and EGF were moderately correlated. Since this study used a multiple analyte assay platform (Luminex), some cross-talk among channels is possible but is unlikely to be the exclusive source of these correlations.

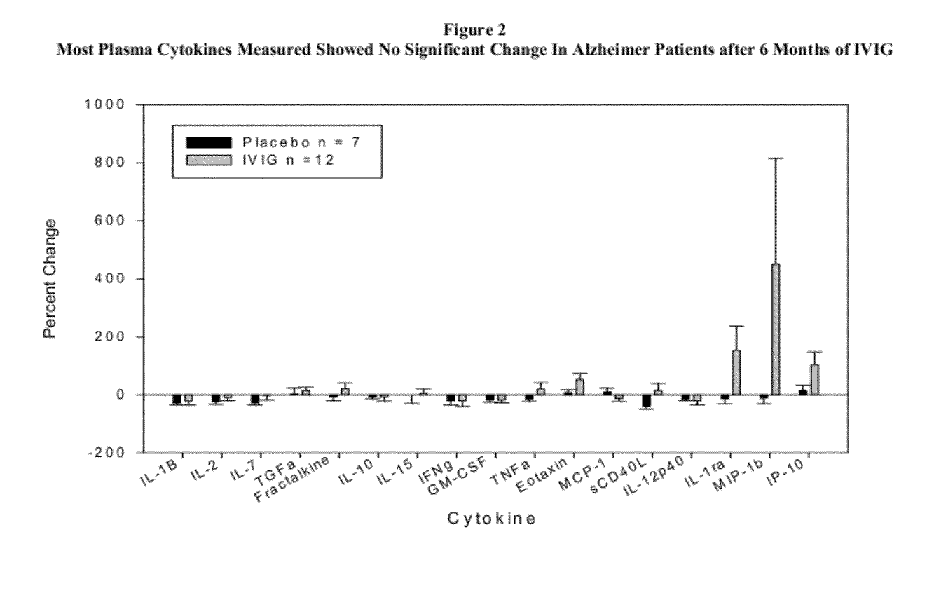

Although most plasma cytokines showed no significant change in AD patients after 6 months of IVIG treatment, a few cytokines including IL-1ra, MIP-1B, and IP-10 demonstrated notable changes, i.e., a notable increase from their corresponding level observed in untreated control subjects (see FIG. 2).

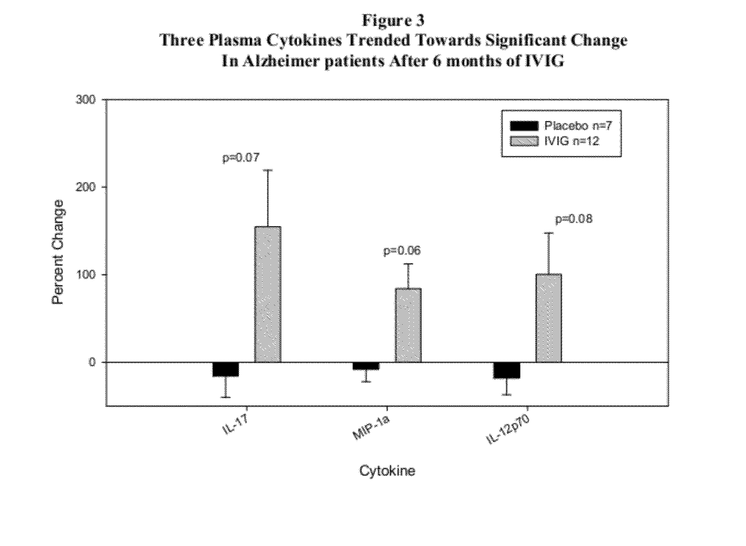

In this study three plasma cytokines, IL-17, MIP-1a, and IL-12p70, showed trend towards significant change in AD patients after 6 months of IVIG treatment (FIG. 3).

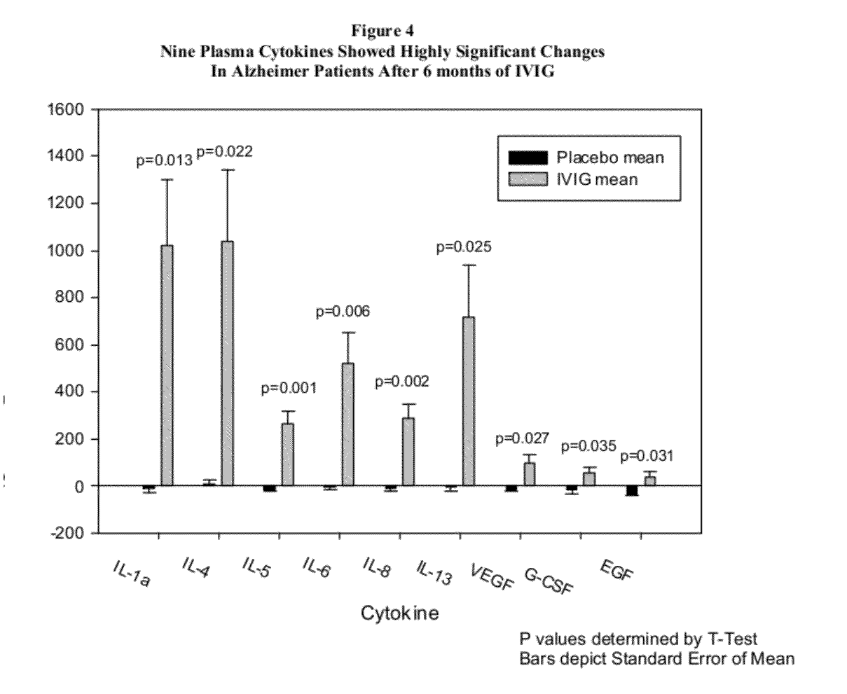

Highly significant changes in plasma level of nine cytokines were observed in AD patients after 6 months of IVIG-treatment: IL-1A, IL-4, IL-5, IL-6, IL-8, IL-13, VEGF, G-CSF, and EGF (see FIG. 4).

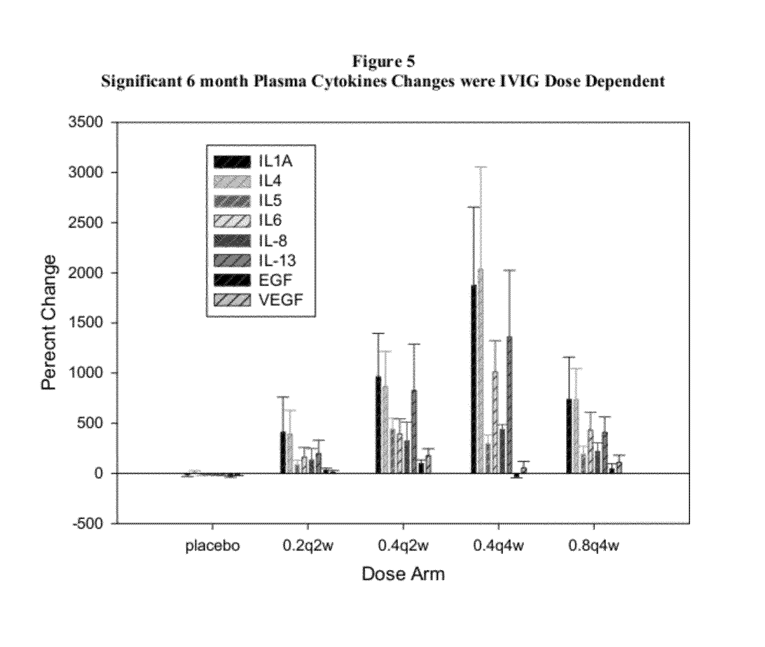

Another observation made during this study is that the significant changes in plasma levels of cytokines IL1A, IL4, IL5, IL6, IL8, IL13, EGF, and VEGF after 6 months of IVIG treatment were in an IVIG dose dependent manner (see FIG. 5).

Furthermore, correlation between clinical outcomes and plasma cytokine measurements was established in this study. In global outcome at 6 months, CGIC score correlated modestly (r=0.32) with IL-13 levels. A stronger correlation was observed with G-CSF (r=0.74) among 11 subjects evaluated for the cytokine.

In cognitive outcomes at 6 months, MMSE change scores showed a modest positive correlation with change in plasma levels of IL-8 (r=0.45). 3MS change score correlated positively with IL-5 (r=0.45) and IL-6 (r=0.45). ADAS-Cog were correlated with G-CSF, TNF-alpha, and Eotaxin levels, but the latter two were not among the cytokines that changed significantly after IVIG treatment versus placebo.

In behavioral outcome at 6 months, the NPI results correlated modestly with IL-8 (r=0.32) and IL-5 (r=0.31).

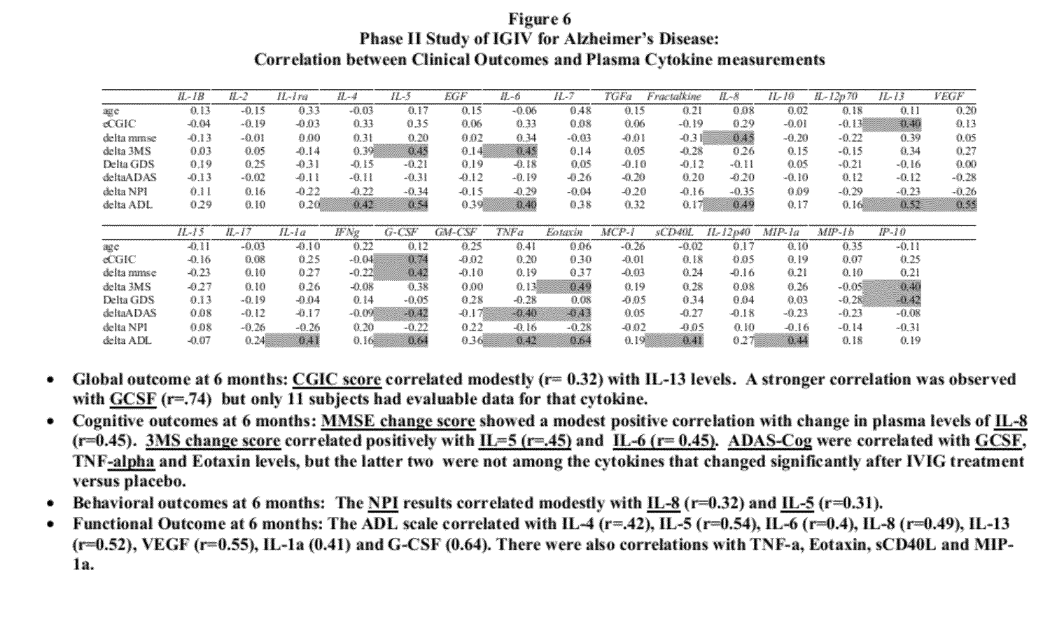

In functional outcome at 6 months, the ADL scale correlated with IL-4 (r=0.42), IL-5 (r=0.54), IL-6 (r=0.4), IL-8 (r=0.49), IL-13 (r=0.52), VEGF (r=0.55), IL-1a (r=0.41), and G-CSF (r=0.64). There were also correlations with TNF-alpha, Eotaxin, sCD40L, and MIP-1a. The correlations between clinical outcome and plasma cytokine levels are shown in FIG. 6.

### CONCLUSION

The expression of a specific set of plasma cytokines changed significantly following 6 months of IVIG infusions in subjects with AD. These cytokines include IL-1A, IL-4, IL-5, IL-6, IL-8, IL-13, GCSF, EGF, and VEGF. Changes in three other cytokines, IL-17, MIP-1A, and IL-12P70, showed trends towards significance. These changes were IVIG dose dependent: only minor changes occurred over time in the placebo group; among subjects receiving IVIG, the numerically smallest changes were seen with IVIG 0.2 g/kg/2 week, but even at that dose the changes were substantial. No strong correlations were observed between clinical outcomes and changes in plasma cytokines levels. However, several low to moderate correlations (r=0.3-0.5) were found. IL-5 and IL-8 correlated with cognitive, behavioral, and functional outcomes, whereas IL-13 and GCSF correlated with global outcome.

### DISCUSSION

These findings support the hypothesis that IVIG has immune-modulatory effects in AD patients. IVIG does not contain significant amount of cytokines, so the elevation of plasma cytokine levels observed in this study therefore must represent the distal effects of the antibodies in IVIG rather than an accumulation of exogenous cytokines. The correlation between plasma cytokine changes and clinical outcomes in this study is relatively modest (r=0.3-0.5) but approximates the level of correlation observed between clinical outcomes and CSF anti-amyloid oligomer antibodies (r=˜0.41) in the same subjects. The present results suggest that there may be readily discernible differences in cytokine expression between and placebo, 0.2 g/kg weight/2 wk, and 0.4 g/kg weight/2 wk dose arms.

All patents, patent applications, and other publications, including GenBank Accession Numbers, cited in this application are incorporated by reference in the entirety for all purposes.

What is claimed is:

1. A method for treating Alzheimer's disease in a subject in need thereof, comprising the sequential steps of:
(a) determining the amount of a cytokine in the subject's blood, thereby obtaining a baseline value of the cytokine level;
(b) administering a brain preserving therapeutic agent to the subject for the purpose of treating Alzheimer's disease during a first time period;
(c) determining the amount of the cytokine in the subject's blood, thereby obtaining a first intermediate value of the cytokine level;
(d) comparing the intermediate value from step (c) with the baseline value from step (a); and
(e) increasing administration of the brain preserving therapeutic agent in dose or frequency when step (d) indicates no increase from the baseline value to the first intermediate value, or maintaining administration of the brain preserving therapeutic agent in dose or frequency when step (d) indicates an increase from the baseline value to the first intermediate value.

2. The method of claim 1, wherein steps (b) to (d) are further repeated at least once and in each repeat the latest intermediate value is compared with the second latest intermediate value to determine future administration of the therapeutic agent in the same manner as step (e).

3. The method of claim 1 or 2, wherein step (d) during any repeat indicates no increase from one intermediate value to its subsequent intermediate value, and the administration of the brain preserving therapeutic agent is increased in dose or frequency, further comprising the steps of:
(f) determining the cytokine level in the subject's blood after an additional time period during which the therapeutic agent is administered to the subject, thereby obtaining additional intermediate value of the cytokine level;
(g) comparing the additional intermediate value with its previous intermediate value; and
(h) discontinuing further administration of the therapeutic agent when step (g) indicates no increase from the previous intermediate value to the additional intermediate value, or maintaining administration of the brain preserving therapeutic agent in dose or frequency when step (g) indicates an increase from the previous intermediate value to the additional intermediate value.

4. The method of claim 1, wherein the first time period is 3 months, 6 months, 9 months, 12 months, or 18 months.

5. The method of claim 1 or 3, wherein the second or subsequent time period is 3 months, 6 months, 9 months, 12 months, or 18 months.

6. The method of claim 1, wherein the cytokine is IL-1A, IL-4, IL-5, IL-6, IL-8, IL-13, VEGF, G-CSF, EGF, IL-12p70, IL-17, MIP-1A, MIP-1B, or IP-10.

7. The method of claim 1, wherein the therapeutic agent is an intravenous immunoglobulin (IVIG) composition.

8. The method of claim 7, wherein the IVIG composition is administered subcutaneously.

9. The method of claim 7, wherein the IVIG composition is administered intravenously.

10. The method of claim 7, wherein the IVIG composition is administered at about 0.2 to 2 grams per kg body weight of the subject per month.

11. The method of claim 7, wherein the IVIG composition is administered once a week, twice a week, once a month, or twice a month.

12. The method of claim 7, wherein the IVIG composition is administered at about 0.4 gram per kg body weight of the subject twice a month.

13. The method of claim 1 or 3, wherein step (a), (c), or (f) is performed by an immunoassay.

14. The method of claim 1 or 3, wherein step (a), (c), or (f) is performed by mass spectrometry.

15. A method for assessing efficacy of a therapy intended for treating Alzheimer's disease, comprising the steps of:
(a) determining the average level of a cytokine in the blood of subjects suffering from Alzheimer's disease but not receiving the therapy, thereby obtaining a non-therapeutic level of the cytokine;
(b) determining the average level of the cytokine in the blood of subjects suffering from Alzheimer's disease and receiving the therapy, thereby obtaining a therapeutic level of the cytokine; and
(c) comparing the therapeutic level with the non-therapeutic level, thereby determining the efficacy of the therapy,
wherein the therapy is deemed effective when the therapeutic level is higher than the non-therapeutic level, and the therapy is deemed ineffective when the therapeutic level is equal to or lower than the non-therapeutic level.

16. The method of claim 15, wherein the cytokine is IL-1A, IL-4, IL-5, IL-6, IL-8, IL-13, VEGF, G-CSF, EGF, IL-12p70, IL-17, MIP-1A, MIP-1B, or IP-10.

17. The method of claim 15, wherein the therapy is administration of an intravenous immunoglobulin (IVIG) composition.

18. The method of claim 17, wherein the IVIG composition is administered subcutaneously.

19. The method of claim 17, wherein the IVIG composition is administered intravenously.

20. The method of claim 17, wherein the IVIG composition is administered at about 0.2 to 2 grams per kg body weight of the subject per month.

21. The method of claim 17, wherein the IVIG composition is administered once a week, twice a week, once a month, or twice a month.

22. The method of claim 17, wherein the IVIG composition is administered at about 0.4 gram per kg body weight of the subject twice a month.

23. The method of claim 15, wherein the cytokine level in step (a) or (b) is determined over a time period of about 3 months, 6 months, 9 months, 12 months, or 18 months.

24. The method of claim 15, wherein step (a) or (b) is performed by an immunoassay.

25. The method of claim 15, wherein step (a) or (b) is performed by mass spectrometry.
